(19) United States
(12) Patent Application Publication
(10) Pub. No.: US 2007/0049837 A1
(43) Pub. Date: Mar. 1, 2007

(54) Acoustic sensor

(76) Inventors: Hemchandra M. Shertukde, Simsbury, CT (US); Peter V. Beckmann, Hartford, CT (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/472,501

(22) Filed: Jun. 21, 2006

Related U.S. Application Data
(60) Provisional application No. 60/692,515, filed on Jun. 21, 2005.

(51) Int. Cl. A61B 5/02

(52) U.S. Cl. 600/528

(57) ABSTRACT

Disclosed are devices, systems, and methods for capturing acoustic data in an efficient manner. Some embodiments have piezoelectric sensing portions with polarization axes and conducting layers. In some embodiments, piezoelectric sensing portions can be positioned generally coplanar to each other and in partial electrical contact. In some embodiments, polarization axes of two piezoelectric sensing portions have a non-zero angle between them. Certain embodiments comprise a noise reducing element. In some embodiments, an electrode is included to detect electrical signals.

### PRIORITY INFORMATION

This application claims priority to U.S. Patent Provisional Application No. 60/692,515, titled “ACOUSTIC SENSOR,” filed Jun. 21, 2005, the entirety of which is hereby incorporated by reference and made part of this specification. This application is related to U.S. patent application Ser. No. 11/415,895, titled “ACOUSTIC SENSOR,” filed May 2, 2006 and U.S. patent application Ser. No. 11/417,952, titled “ACOUSTIC SENSOR,” filed May 3, 2006, both of which are hereby incorporated by reference in their entirety and made part of this specification.

### BACKGROUND OF THE INVENTIONS

1. Field of the Inventions

The inventions described herein relate generally to the field of transducers, and in particular acoustic transducers. For example, some embodiments relate to acoustic sensors that can detect biological sounds and generate accurate data for signal processing to determine biological characteristics relating to the source of those sounds.

2. Description of the Related Art

Transducers are the operative portion of many modern technologies. One useful class of transducers converts an analog signal, such as an acoustic vibration wave, into an electrical signal. In particular, microphones contain acoustic transducers and can detect and record signals that correspond to sounds. The human hear is itself an acoustic transducer.

Designers of acoustic sensors are continually challenged by the problem of separating the desired signal from unwanted noise. This challenge applies to both the acoustic noise (or extraneous acoustic vibrations) as well as the electronic noise (or unwanted electrical signals). Acoustic noise can be distracting background chatter that would be detectable by a human ear, or minute, unheard vibrations caused by a distant truck driving down the street. This kind of noise can interfere with the input of an acoustic sensor. Electronic noise can be electromagnetic emissions that cause the electrons in an electrical device to vibrate or move. This kind of noise can interfere with the output of an acoustic sensor. Because a transducer changes one signal to another signal, it is subject to problems with noise for both types of signals.

Another problem that occurs in current sensors is over-sensitivity to the direction of the signal. For example, in many cases, sensors are structurally capable of effectively detecting signals, but are too sensitive to the orientation of the sensor with respect to the signal. Even relatively small changes in the orientation of the sensor can significantly affect the strength of the received signal, or determine whether the signal is received at all. Thus, many sensors are inefficient because they depend too much on proper orientation. This can lead to repeated tests (if the error is perceived by the operator), or incorrect and unreliable readings.

Another problem of existing sensors relates to the arrival of a signal at various portions of the sensor at different times. For example, in some sensors that have multiple sensing portions that are vertically stacked, one above another, signals arriving from below the stack reach one sensing portion at one time, but that same signal does not reach the other sensing portion until later. This time difference of arrival can create signal time incidence ambiguities in sensor output.

Thus, there is a need for methods and devices for increasing the sensitivity of acoustic transducers, improving shielding, reducing unwanted noise, and enhancing signal to noise ratios. There is also a need for methods and devices for improving the ability of acoustic sensors to receive signals from various directions without requiring time-consuming and error-prone repositioning of the sensors. Moreover, a need exists for improving sensors to minimize problems with the time difference of arrival at various sensing elements and to minimize signal time incidence ambiguities in signal sensor outputs.

### SUMMARY

In one embodiment, an apparatus for sensing acoustic signals from a source comprises a first acoustic sensing element comprising a first piezoelectric portion having a first polarization axis and two electrically conductive portions. The apparatus also comprises a second acoustic sensing element comprising a second piezoelectric portion having a second polarization axis and two electrically conductive portions. The apparatus further comprises a noise reducing element. The apparatus is configured such that one electrically conductive portion of the first piezoelectric portion is electrically connected to one electrically conductive portion of the second piezoelectric portion. The apparatus is further configured such that the first and the second piezoelectric portions are oriented such that the first and the second polarization axes form a non-zero angle therebetween.

Another embodiment of an apparatus for sensing acoustic and electric signals comprises a first acoustic sensing element comprising a first piezoelectric portion having a first polarization axis and two electrically conductive portions and a second acoustic sensing element comprising a second piezoelectric portion having a second polarization axis and two electrically conductive portions. The apparatus further comprises an electrode electrically insulated from the electrically conductive portions of the first and the second acoustical sensing elements. The apparatus is configured such that one electrically conductive portion of the first piezoelectric portion is electrically connected to one electrically conductive portion of the second piezoelectric portion. The apparatus is further configured such that the first and the second piezoelectric portions are oriented such that the first and the second polarization axes form a non-zero angle therebetween. Some embodiments of this apparatus further comprise a noise reducing element.

In another embodiment, an acoustic sensing device comprises a first piezoelectric sensing portion having a first polarization axis and two conducting layers, and a second piezoelectric sensing portion having a second polarization axis and two conducting layer. In this device, the second piezoelectric sensing portion is positioned generally coplanar to the first piezoelectric sensing portion, with one conducting layer of the first piezoelectric sensing portion in electrical contact with one conducting layer of the second piezoelectric sensing portion. The first and second polarization axes have a non-zero angle between them.

An embodiment of a method of manufacturing an acoustic sensor comprise providing a first piezoelectric layer having a first polarization axis and providing two conductive layers, one on either side of the first piezoelectric layer. The method further comprises providing a second piezoelectric layer having a second polarization axis and two conducting layers, one on either side of the second piezoelectric layer. The second piezoelectric layer is positioned generally coplanar to the first piezoelectric layer, with one conducting layer of the first piezoelectric sensing portion in electrical contact with one conducting layer of the second piezoelectric sensing portion. The first and second polarization axes have a non-zero angle between them.

### BRIEF DESCRIPTION OF THE DRAWINGS

Certain embodiments of the inventions will now be briefly described with reference to the drawings. These drawings are examples and the inventions are not limited to the subject matter shown or described.

FIG. 1 is a schematic, perspective view of a sensing layer component of a sensor in accordance with one embodiment of the inventions.

FIG. 2 is a schematic, cross-sectional side view of two sensing layer components, taken along the lines 2-2 of FIG. 3.

FIG. 3 is a schematic plan view of the sensing layer components of FIG. 2 with electrical leads and other components.

FIG. 4 is a schematic, partial cross-sectional side view (taken along lines 44 of FIG. 5) of a portion of a sensor in accordance with one embodiment of the inventions.

FIG. 5 is a schematic perspective view of a sensor in accordance with one embodiment of the inventions.

FIG. 6 is a schematic illustration of multiple sensors positioned on the surface of a patient's chest with electrical leads transmitting data to a processor.

FIGS. 7A-7C are schematic illustrations of certain concepts relating to piezoelectric polarity and electric charges induced by bending of piezoelectric materials.

FIGS. 8A-8B are schematic illustrations of multi-dimensional bending of planar materials and corresponding vector principles.

FIG. 9A is a schematic, cross-sectional illustration of a cut-away side view of a sensor in accordance with one embodiment of the inventions positioned on the skin of a patient, and a point source emitting substantially spherical sound waves.

FIG. 9B is a schematic, cross-sectional illustration of the sensor, point source, and sound waves of FIG. 9A at a later instant in time.

FIG. 9C is a schematic, three-dimensional, elevational illustration of the sensor, point source, and sound waves of FIG. 9B.

FIG. 10 is a perspective view of one alternative embodiment of a sensor in accordance with the inventions.

FIG. 11A is a perspective view of another alternative embodiment of a sensor in accordance with the inventions.

FIG. 11B is a plan view of the sensor of FIG. 11A.

FIG. 11C is a schematic, cross-sectional side view of the sensor of FIG. 11A, taken along the lines 11C-11C of FIG. 11B.

FIG. 12A is a schematic electronic circuit diagram illustrating the effective electrical properties of one embodiment of a sensor with two sensing layers.

FIG. 12B is a schematic electronic circuit diagram illustrating electrical apparatus that can be attached to the circuit of FIG. 12A for testing and/or data processing.

FIG. 12C is a schematic electronic circuit diagram illustrating the effective electrical properties of another embodiment of a sensor with two sensing layers.

FIG. 13A is a schematic, partial cross-sectional side view of a portion of an embodiment of a noise reducing acoustic sensor.

FIG. 13B is a schematic, partial cross-sectional side view of another embodiment of a noise reducing acoustic sensor.

FIG. 14 is a schematic, partial cross-sectional side view (taken along the line 14-14 of FIG. 15) of a portion of a combined acoustic and electrical sensor.

FIG. 15 is a schematic perspective view of a combined acoustic and electrical sensor in accordance with one embodiment of the inventions.

### DETAILED DESCRIPTION OF THE PREFERRED EMBODIMENTS

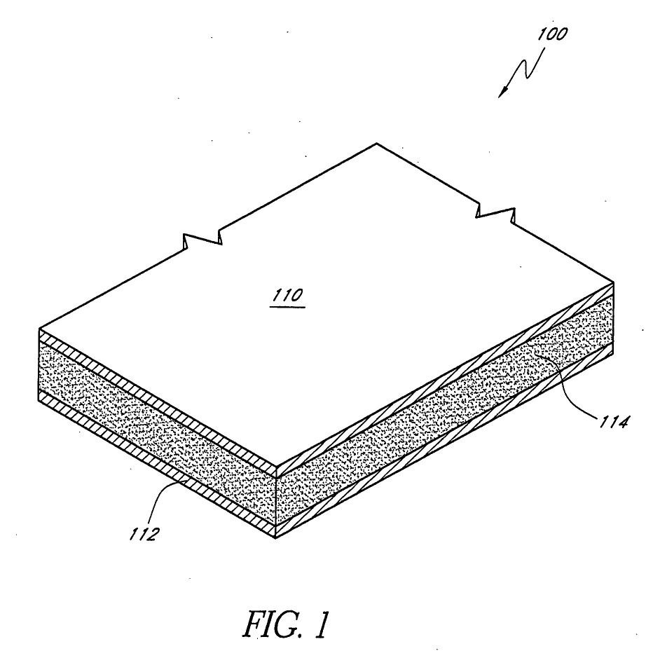

With reference to FIG. 1, a sensing layer 100 comprises a piezoelectric central portion 114 with a top conductive layer 110 and a bottom conductive layer 112. The piezoelectric material can be a piezoelectric co-polymer. In one advantageous embodiment, the piezoelectric portion 114 is formed from polyvinylidene fluoride (PVDF). PVDF is an anisotropic piezoelectric polymer that produces surface charges of substantially equal magnitude and opposite polarity on opposite surfaces when a mechanical strain is imposed on the material. Other preferred materials that may form the sensing portions can comprise compounds that include vinylidene fluoride. One preferred material is a piezoelectric co-polymer that is 75% vinylidene fluoride by weight. Metallized PVDF can be obtained from Measurement Specialties, Inc., of Hampton Va. 23666. PVDF can be used to good effect due to its compliance and resilience, as well as its piezoelectric properties. Moreover, PVDF is inexpensive, is the most commonly used commercial piezoelectric polymer, and has properties which are relatively unaffected by synthesis conditions. Other piezoelectric materials can also be used, such as poly vinylidene cyanide and its copolymers; aromatic and aliphatic polyureas; poly vinyl chloride; aromatic polyamides (odd nylons); PVDF copolymers with trifluoroethylene (P[VDF-TrFE)), tetrafluoroethylene (P[VDF-TFE)), and hexafluoropropylene (P[VDF-HFP)); PVDF blends with poly methyl methacrylate (PMMA); poly vinyl fluoride; poly vinyl acetate; and ferroelectric liquid crystal polymers.

As illustrated schematically, the piezoelectric portion 114 is not completely surrounded by the metallized portions (top conductive layer 110 and bottom conductive layer 112). Preferably, the top conductive layer 110 and the bottom conductive layer 112 are not in electrical contact with each other when the sensing layer is in the illustrated configuration. Neither the sensing layer 100, nor its sub-layers (the top and bottom conductive layers 110 and 112 and the piezoelectric portion 114) are shown to scale in FIG. 1.

The conducting layers 110 and 112 can comprise metallization layers that are adhered to the surfaces of the piezoelectric portion 114. The conducting layers 110, 112 can adhere to the surface of the piezoelectric portion by any suitable process, such as a deposition process. Metallization of the surfaces of the piezoelectric portion 114 may be accomplished using any suitable material and any suitable technique known in the art. For example, thin layers of a metal, such as nickel, silver, copper or alloys thereof, can be deposited on the inner and outer surfaces of the sensing layer 114. In other embodiments, the conductive layers 110 and 112 can comprise or be coated with a conducting ink.

In a preferred embodiment, the piezoelectric portion 114 is preferably thicker than either of the two conducting layers 110, 112. In some embodiments, the piezoelectric portion 114 has a thickness of about 100 microns or less. In certain preferred embodiments, the piezoelectric portion 114 has a thickness of less than 150 microns, and the top and bottom conducting layers 110 and 112 each have a thickness of less than 30 microns. In one preferred configuration, the sensing layer 100 comprises PVDF with a copper nickel alloy coating. The piezoelectric portion can have a thickness between approximately 150 μm and approximately 6 μm. In some preferred embodiments, the total thickness of the piezoelectric portion 114 and the two conducting layers 110 and 112 combined is approximately 28 μm.

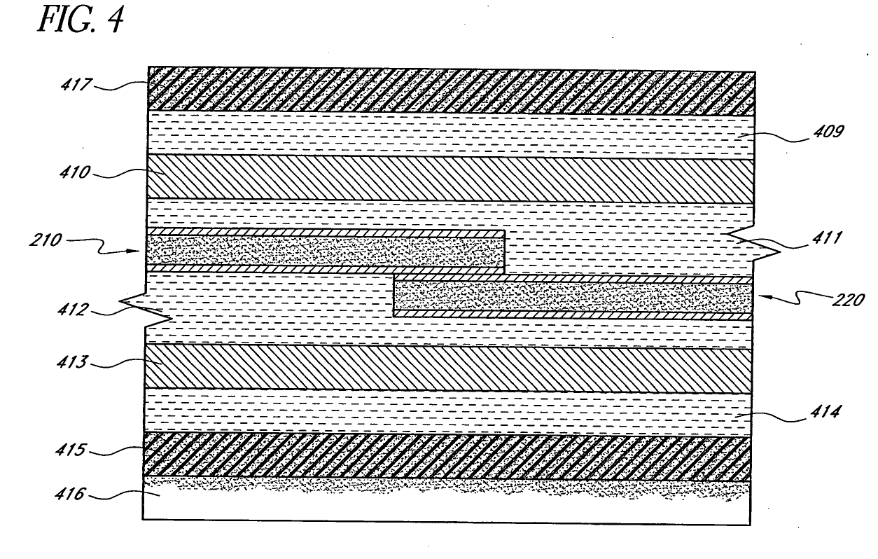

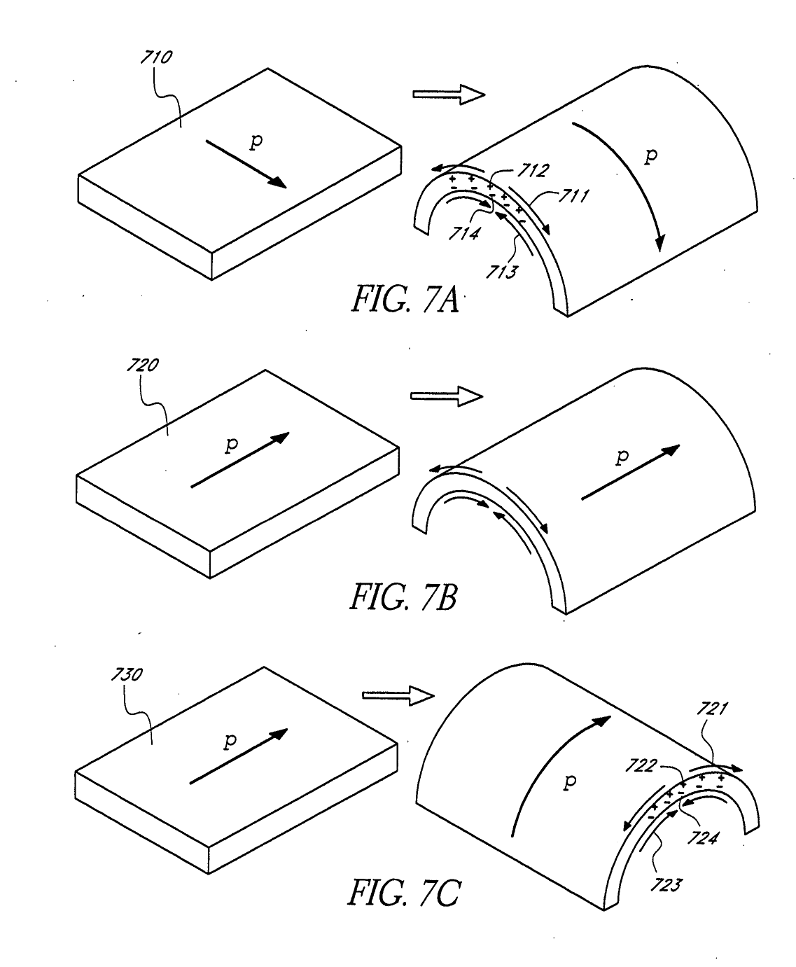

Typically, when a tensile strain is imposed on the piezoelectric portion 114, one surface of the piezoelectric portion 114 acquires a positive charge relative to the other. The charge is typically transferred to one of the adjacent conductive layers 110 or 112. The piezoelectric portion 114 is advantageously polarized such that the piezoelectric effect is greater when the piezoelectric portion is stretched in a particular direction. The polarization axis can also be referred to as the “stretch axis.” As used herein, the terms “polarization axis,” “polarization,” or “polarized” refer to the directional dependence of the piezoelectric response of a piezoelectric material in the plane in which the material is stretched. Although biaxial orientation (stretching in two in-plane dimensions) is possible, it produces piezoelectric films with lower bilaterally isotropic piezoelectric properties. Most commercially available PVDF is uniaxially drawn, providing a high level of piezoelectric response along the stretch axis, or axis of orientation. The stretch axis, which is interchangeably referred to herein as the polarization axis, is denoted by an arrow referenced with the letter “P” in the drawings (e.g., see FIGS. 3, 7A-7C, etc.). It is recognized that a piezoelectric layer comprises material having a dipole moment that is generally perpendicular to the “stretch plane” (e.g., parallel or antiparallel to a direction between the layers 110 and 112 in FIG. 1). In some piezoelectric materials, the direction of the dipole moment is generally the same as the poling direction during fabrication of the piezoelectric material. As used herein, the terms “polarization axis,” “polarization,” and “polarized” do not refer to the direction of the dipole moment or the poling direction.

When the piezoelectric portion 114 is under strain, the oppositely polarized charge that accumulates on the opposite layers of the piezoelectric portion spreads out over the top and bottom conductive layers 110 and 112, forming a capacitive effect between the two conductive layers 110 and 112. Because of this configuration, the voltage as measured across the two conductive layers 110 and 112 is related through the capacitance equation: Q=CV, where Q is the amount of surface charge, C is the capacitance, and V is the voltage output. Q can be expressed in Coulombs, C can be expressed in Farads, and V can be expressed in Volts. Certain configurations of PVDF materials exhibit a predictable voltage output V in response to a specific applied force. Generally, the amount of surface charge Q is proportional to the strain on the piezoelectric material, and capacitance C is substantially constant for a given material and structure. Thus, both Q and V are generally proportional to the strain on the piezoelectric material. If the voltage or charge response function is known, a measurement of either parameter can provide information about the strength of the signal (e.g., acoustic vibration) causing the strain. Moreover, if the precise response function of the piezoelectric material for a given physical configuration is not known, the output voltage can still provide useful data because the responses at various times can be compared.

Furthermore, if the piezoelectric portion is polarized, information relating to the direction of the acoustic energy can also be obtained. Alternatively, a combination of two piezoelectric portions that are polarized in different directions can be configured to provide accurate data regarding the magnitude of the sensed signal, independent of the signal direction upon arrival at the sensor.

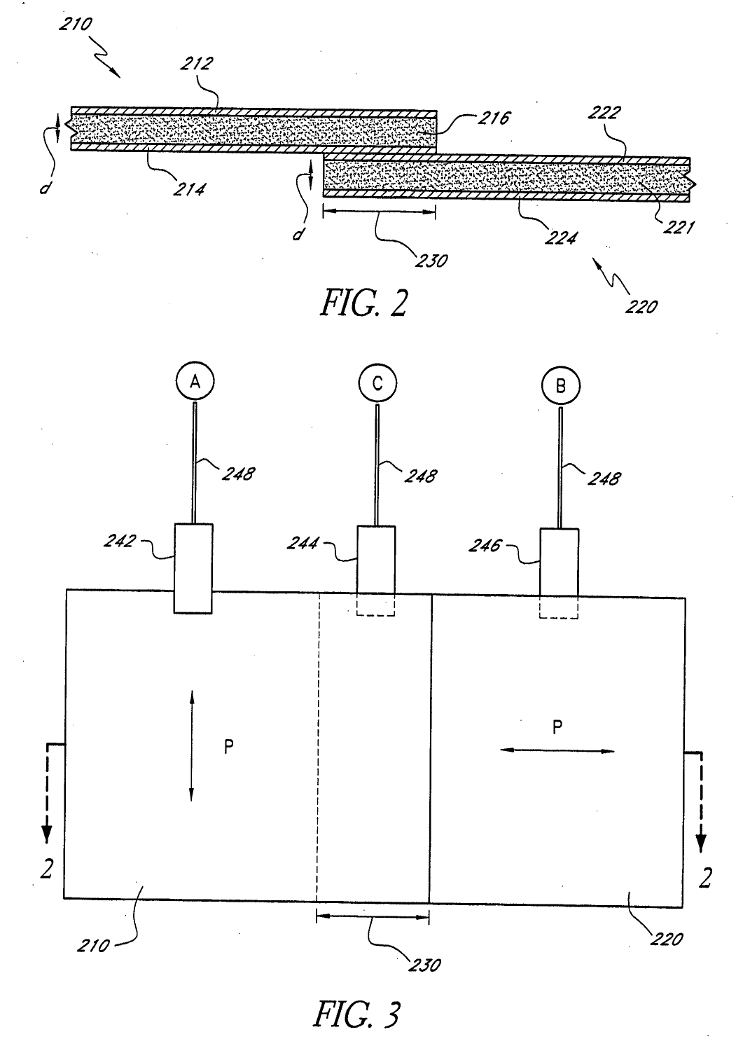

With reference to FIG. 2, two sensing layers 210 and 220 (each similar to the sensing layer 100 described above) are positioned in a generally parallel, partially displaced, and generally coplanar orientation. While the two sensing layers 210 and 220 are not precisely coplanar in the illustrated embodiment, they are only vertically shifted by the width “d” of a single sensing portion, which can be less than 100 microns, as discussed above. In FIG. 2, the two sensing layers 210 and 220 are schematically illustrated in cross section. The sensing layer 210 has a top conductive layer 212 and a bottom conductive layer 214. The sensing layer 220 has a top conductive layer 222 and a bottom conductive layer 224. In a preferred configuration, the conductive layers 214 and 222 are in electrical contact and are configured to receive charges of the same polarity from their respective piezoelectric portions (the layer 214 receives charge when the layer 216 is appropriately stressed, and the layer 222 receives charge from the layer 221 under the appropriate stress). For example, if the bottom conductive layer 214 accumulates negative charge when the piezoelectric portion 216 is under strain, the top conductive layer 222 accumulates negative charge when the piezoelectric portion 221 is under strain. Similarly, the outer two conductive layers, 212 and 224, accumulate charge of the same polarity.

The described configuration, where the sensing layers 210 and 220 are “inverted” with respect to each other (that is, configured to have charge of opposite polarity accumulate on the top and bottom layers of the two sensing layers, respectively), provides the advantage of allowing a single electrical lead to contact two conductive layers. (The electrical lead 244 is in contact with both the conductive layers 214 and 222. See FIG. 3). In another preferred embodiment, the sensing layers 210 and 220 are not inverted with respect to each other (e.g., they are configured to have charge of the same polarity accumulate on the top layers of the two sensing layers). Such embodiments may advantageously provide improved signal-to-noise in comparison to the “inverted” embodiments.

With reference to FIG. 3, a plan view of the two sensing layers 210 and 220 is illustrated schematically. In this figure, the polarization of the two sensing layers 210 and 220 is shown with the two-sided arrows and the letters “P.”, Thus, the sensing layer 210 is polarized substantially orthogonally to the sensing layer 220. The orthogonally polarized sensing portions 210 and 220 provide a multi-directional sensing capability. For example, a signal that bends the sensing portion 210 in such a way that little or no electrical response is produced in that portion will have a higher likelihood of bending the sensing portion 220 in such a way that an electrical response will be produced in that portion. Indeed, as explained further below, the two mechanically coupled but oppositely polarized sensing portions 210 and 220 can act together to sense any arriving signal. Moreover, those vector components that are less likely to be sensed by the sensing portion 210 are more likely to be sensed by the sensing portion 220.

Some embodiments have two polarized sensing portions where the polarization directions of the sensing portions are not orthogonal, but are non-parallel, having a relative angle of anywhere between zero and ninety degrees. Sensing portions that are not polarized parallel to each other can be used to sense incoming signals from multiple directions. Furthermore, the relative angle can be chosen to provide the sensor with direction-identification capabilities, or with more efficient magnitude sensing capabilities.

With further reference to FIG. 3, the two sensing layers 210 and 220 overlap by an overlap distance 230. In some embodiments, the overlap distance 230 is approximately 3 mm. The overlap between the sensing layers 210 and 220 provides an electrical connection, as discussed above, as well as a mechanical connection. The combination of the overlap distance 230 and the similar properties of the two sensing portions 210 and 220 can result in the coupled system approximating the behavior of a single plane. For example, the physical dimensions (width thickness, etc.) and characteristics (stiffness, elasticity, tensile strength, etc.) of the two sensing portions 210 and 220 are typically similar, because in some embodiments the two sensing portions have been cut from the same type of material. For example, the two sensing portions 210 and 220 can be cut using a template from a single sheet of stock PVDF. In some embodiments, the two sensing portions 210 and 220 have the same dimensions but are cut in different orientations with respect to the polarization of the stock PVDF. Furthermore, the thickness “d” of the two sensing portions 210 and 220 is typically small compared to the other dimensions of the sensing portions 210 and 220, and the dimensions of the combined system are many times greater than either the thickness “d” or the overlap distance 230. Some preferred embodiments have sensing portions, each having following dimensions: 14.2 mm×30 mm and a thickness “d” of approximately 28 μm.

Preferably, the two sensing portions 210 and 220 have enough overlap 230 to remain mechanically coupled and electrically linked, but not so much overlap that the resilience of the planar system is significantly altered. Thus, the two sensing layers 210 and 220 can physically bend and respond much the same way a continuous plane of the same material would respond to an impinging acoustic signal. Some preferred embodiments have an overlap distance 230 of 3 mm. For example, when the sensing portions are 14.2 mm×30 mm, the overlap 230 can occur along the 30 mm length of the two sensing portions 210 and 220. In this configuration, the total area of the sensor can be approximately 762 mm².

The illustrated configuration also has the advantage of allowing impinging acoustic signals to arrive at the two sensing layers 210 and 220 essentially in unison—that is, such that the time difference of arrival (TDOA) is minimal. Thus, in some embodiments, configurations described herein can be referred to as “iso surface optimal material adherent compliant,” or “ISOMAC” sensors. The two sensing layers with optimized areas can lie in the same plane, thus generally presenting an “iso surface,” or a surface at which various points lie generally at the same distance from the source of the impinging acoustic signal. Moreover, as described further below, the materials from which a sensor is constructed can be compliant to the skin surface, bending in response to an impinging acoustic signal, while at the same time adhering to the surface of the skin to allow efficient mechanical coupling.

In the illustrated embodiment, the electrical lead 242 is in electrical contact with the conductive layer 212. The electrical lead 244 is in contact with both the conductive layers 214 and 222. The electrical lead 246 is in contact with conductive layer 224. In some embodiments, the electrical leads 242, 244, and 246 can comprise metal lugs, each having a 5mm lip. Other ways of making electrical connections can also be used. As illustrated, the leads 242, 244, and 246 each attach to a shielded pair of twisted wires 248. Because each pair is similar in the illustrated embodiment, each pair of wires has been labeled 248 in FIG. 1. One way to connect the leads 242, 244, and 246 to the wires 248 is by soldering or crimping. Electrical connections can also be formed using an EC adhesive.

Electrical lead 242 corresponds to the A terminal, electrical lead 244 corresponds to the C terminal, and electrical lead 246 corresponds to the B terminal. A and B can be positive terminals, while C is a “common,” or ground terminal. Alternatively, A can be a positive terminal, while C is a ground terminal, and B is a negative terminal. Various electrical connections can be made to measure the voltage difference across the sensing layers 210 and 220.

The electrical leads 242, 244, and 246 provide a way for the charge that accumulates on the conductive layers 212, 214, 222, and 224 to be measured using an electrical circuit and connections that will be described further below. If the physical properties of the sensing portions 210 and 220 are known, the equations and physical relations described herein can allow for the calculation of the acoustic signal's magnitude, direction, etc. The charge that accumulates on the top and bottom surfaces of the piezoelectric portions encounters relatively little resistance in the conductive layers 212 and 214, 222, and 224. Thus, the charge present on any particular layer can be measured at and/or collected from any contact point on that layer.

In a preferred embodiment, the sensing portions 210 and 220 can be modeled using the physical electrostatic equations for two-plate capacitors. For example, in the illustrated configuration, the two sensing portions 210 and 220 have equal area. This configuration is generally analogous to two displaced capacitors with opposite electrical orientations. Assuming that the conductive layers 214 and 222 that are in contact have a negative charge, the capacitance and voltage of the two elements individually can be described thus, after charge has accumulated:
Cⱼ=(Aⱼε)/dⱼ . . . for j=1,2, (1)
where Cⱼ is the capacitance of the ‘j’th element, Aⱼ is the area of the ‘j’th element, ε is the electrical permittivity of the medium (e.g., PVDF), and dⱼ is the separation between the conductive layers of the ‘j’th element (which corresponds to the widths of the piezoelectric portions 216 and 226), or the separation distance between the parallel plates of the capacitor. In a preferred embodiment, d₁=d₂=28 μm. Thus the corresponding voltage signal generated at the two plates of any element is
Vⱼ=Qⱼ/Cⱼ . . . for j=1,2 (2)

Assuming that the conductive layers 212, 214, 222, and 224 each have equal area, (A₁=A₂), that each piezoelectric portion 216 and 226 have the same thickness, (d is constant), and that the piezoelectric portions are formed from the same material (and thus are equal in electrical permittivity), (ε is constant), it follows from equations (1) and (2) above that
C₁=C₂=C (3)

Thus, using the capacitance equation for charge ‘Q’ in coulombs given by
Q=CV (4)
it follows that
V₁=V₂=V (5)

Thus, under the assumptions outlined above, one can optimize the physical configuration as needed. For example, multiple elements can be arranged or the surface area of the elements can be expanded to increase the voltage output of the sensor. That is, if ε and d are known and held constant, A can be varied or optimized in order to optimize or maximize Q and V. Alternatively, A, d, j, and/or ε can be varied in order to achieve a desired Q or V. One of the characteristics of some of the embodiments described herein is a design where the sensor is sized to be placed on the skin over the intercostals muscles without significantly overlapping the ribs. The sensor can also be designed to fit within an adhesive envelope of a given size. One such envelope requires a sensor to be less than 1 inch×1 inch, for example. Thus, the sensor area may have a certain maximum value. Voltage output can also be engineered to fall within a certain range under any constraints of electronic hardware. For example, in some embodiments, preferred voltage output is between approximately 0 and approximately 5 volts. The desired gain, dynamic range, and other characteristics of the electronics into which the voltage signal will travel can all provide design parameters. Some embodiments achieve adequate signal strength under these parameters with a total sensor area of approximately 762 mm², for example.

FIG. 4 depicts the structure of FIG. 2 after more materials have been added in layers. As shown, the sensing layers 210 and 220 are positioned between metallic shielding layers 410 and 413 (that can, for example, be formed from a metal such as aluminum). The layers 410 and 413 have been attached to the sensing portions 210 and 220 using layers of adhesive material 411 and 412. The adhesive material that forms the layers 411 and 412 is preferably a flexible material that is not electrically conductive. An electrical connection can be made between both of the metallic shielding layers 410 and 413, and terminal C (the common, or ground terminal). To make such a connection, a flexible, electrically conductive adhesive can be used. A flexible adhesive material 409 and 414 that is not electrically conductive can be used to attach two electrically insulating compliant membranes 415 and 417, as illustrated. One material that can be used to form the compliant membranes 415 and 417 is silicone. A layer of biocompatible adhesive 416 can be placed on one of the compliant membranes 415 or 417. In the illustrated embodiment, the biocompatible adhesive 416 has been placed on the compliant membrane 415. One material that can be used to form the biocompatible adhesive is “Ludlow Hydrogel,” available from Ludlow, a division of Tyco Healthcare Group LP, Chicopee, Mass., 01022.

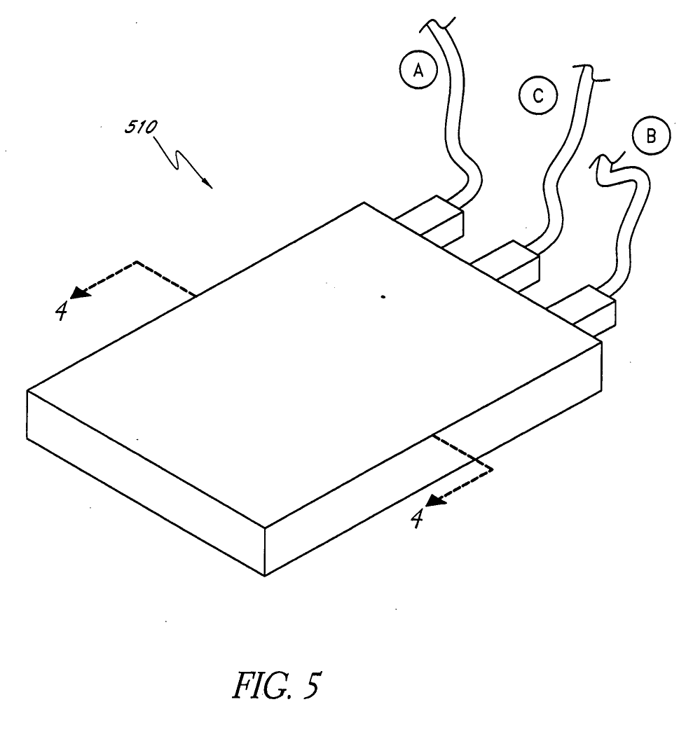

The metallic shielding layers 410 and 413 can provide an electrical shielding effect to minimize unwanted electrical noise. Thus, they can form a continuous or substantially continuous conducting surface that prevents stray electrical charges from penetrating inside the shielding layers 410 and 413. In some embodiments, the metallic shielding layers can be formed from discontinuous mesh that provides shielding. The shielding layers 410 and 413 preferably flex with the other layers, allowing the acoustic signal to freely deform the sensor 510 (see FIG. 5). Thus, the shielding layers 410 and 413 preferably provide a Faraday cage to electrically isolate the sensing portions 210 and 220, while at the same time not unduly stiffening the sensor or having a decisive influence on its over-all mechanical impedance. However, the stiffness and resiliency of the shielding layers 410 and 413 can be selected to provide a portion of the mechanical impedance such that the overall impedance matches that of the surface of human skin, for example.

The flexible, electrically non-conductive adhesive material that forms the layers 411 and 412 preferably provides a permanent connection between the sensing portions 210 and 220 and the shielding layers 410 and 413. The layers 411 and 412 preferably flex readily when acoustic signals impinge on the sensor 510, operating to mechanically couple the layers without contributing to the electrical response. The layers 411 and 412 also preferably insure that no charge passes from the sensing layers 210 and 220 to the shielding layers 410 and 413. The layers 411 and 412 are preferably uniformly distributed, having very few irregularities or discontinuities. Furthermore, the layers 411 and 412 preferably adhere smoothly and evenly to the surfaces they contact.

The compliant membranes 415 and 417 can provide a protective, water repellant layer that protects the electrical connections inside the compliant membranes 415 and 417 from unwanted moisture. In some embodiments, the compliant membranes 415 and 417 form a continuous outer layer that surrounds all other layers except the biocompatible adhesive 416. The compliant membranes 415 and 417 can also have a mechanical impedance that corresponds to that of human skin, for example. The compliant membranes 415 and 417 can thus continuously conform to the changing contours of the surface of human skin as the skin responds to impinging acoustic energy. The compliant membranes 415 and 417 help keep the acoustical loss between the skin and the sensor at a minimum. The described configuration can provide for good sensor sensitivity by using a silicone compliant material to interface with the skin surface.

The flexible, electrically non-conductive adhesive material that forms the layers 409 and 414 preferably provides a permanent connection between the shielding layers 410 and 413, and the electrically insulating compliant membranes 415 and 417. The layers 409 and 414 preferably flex readily when acoustic signals impinge on the sensor 510, operating to mechanically couple the layers without contributing to the electrical response. The layers 409 and 414 also preferably help insure that no charge passes between the outside of the sensor 510 and the shielding layers 410 and 413. The layers 409 and 414 are preferably uniformly distributed, having very few irregularities or discontinuities. Furthermore, the layers 409 and 414 preferably adhere smoothly and evenly to the surfaces they contact.

In some embodiments, a biocompatible adhesive 416 is used to improve the mechanical and acoustic connection between the compliant membrane 415 and skin. The biocompatible adhesive 416 can be “Hydrogel,” (as previously described), which can be positioned at the skin-sensor interface to improve sensitivity and acoustic/mechanical coupling. In some embodiments, the biocompatible adhesive 416 is smeared onto the human skin surface where the sensor 510 will be placed, and the sensor 510 is pressed onto the same area of the skin. The adhesive 416 can also be placed on the sensor 510 before it is pressed into place. In some embodiments, the biocompatible adhesive 416 is located beneath a removable strip (not shown) on the sensor when the sensor 510 is packaged, and the user can remove the strip to reveal the biocompatible adhesive 416 underneath, immediately prior to using the sensor 510.

As schematically illustrated in FIG. 5, the layers described above can form a sensor 510 and leads that correspond to terminals A, C, and B, as described above. As illustrated, FIG. 4 shows a schematic, cross-sectional view of the sensor 510 taken along lines “4-4.”

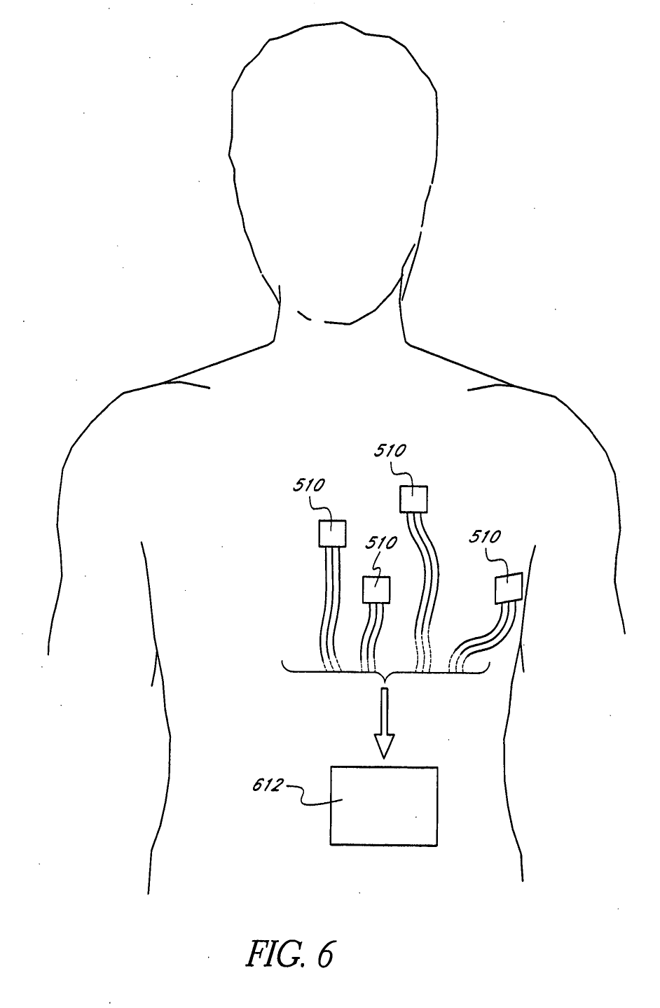

With reference to FIG. 6, the described sensors can be advantageously employed in a system for detecting and processing heart sounds, such as that described in U.S. patent application Ser. No. 10/830,719, filed Apr. 23, 2004, published Feb. 17, 2005, and U.S. patent application Ser. No. 11/333,791, filed Jan. 17, 2006, each of which is hereby incorporated by reference in its entirety and made part of this specification. The improved sensing abilities of the sensors 510 can help enable the accurate detection and localization of stenoses in portions of a human heart, for example. In some embodiments, multiple sensors can be employed to collect data from a plurality of locations on a human body surrounding a human heart, for example. In particular, FIG. 6 schematically shows one possible configuration of multiple sensors 510 and their general placement on a human body. In some embodiments, four sensors can be positioned on the surface of the skin, generally on the external anatomy surrounding the human heart. The sensors 510 can collect acoustic data from sounds emanating from within the body (e.g., the coronary artery). For example, the sensors 510 can gather data for the same acoustic signal from multiple spatial points. The sensors can electronically communicate with a signal processing system 612, conveying, for example, electrical signals that convey information relating to acoustic signals. Examples of such a signal processing system are described in U.S. patent application Ser. No. 10/830,719 and U.S. patent application Ser. No. 11/33,791. The described sensors can aid in the clinical benefits described by providing accurate acoustic data, whatever the arrival direction of the acoustic signal.

With reference to FIG. 7A, a polarized piezoelectric slab 710 is illustrated. The slab 710 undergoes a strain in the polarization direction, resulting in an accumulation of charge at the surfaces of the slab. The piezoelectric material at the top of the slab 710 undergoes tensile forces 711 in the polarization direction, which results in an accumulation of positive charge at the upper surface. In contrast, the piezoelectric material at the bottom of the slab 710 undergoes contractive forces 713 in the polarization direction, which results in an accumulation of negative charge at the lower surface.

FIG. 7B illustrates schematically how a polarized piezoelectric slab 720 that undergoes a strain in a direction orthogonal to its polarization does not accumulate significant charge at its surfaces. Thus, the single polarized piezoelectric slab 720 does not normally sense input effectively if that input does not cause a strain that is aligned with polarization of the slab 720. The piezoelectric material at the top of the slab 720 is subjected to the same tensile forces as the slab 710 illustrated in FIG. 7A, and the piezoelectric material at the bottom of the slab 720 undergoes the same contractive forces as the slab 710. However, because the slab 720 is polarized orthogonally, no piezoelectric effect is produced in the slab 720.

In contrast, FIG. 7C illustrates how the same polarized piezoelectric slab 720 can accumulate charge, and thus effectively “sense” a signal that causes a differently oriented force on the slab 720. In particular, the piezoelectric material at the top of the slab 720 undergoes tensile forces 721 in the same direction of polarization, which results in an accumulation of positive charge 722 at the upper surface. In addition, the piezoelectric material at the bottom of the slab 720 undergoes contractive forces 723 in the same direction of polarization, which results in an accumulation of negative charge 724 at the lower surface.

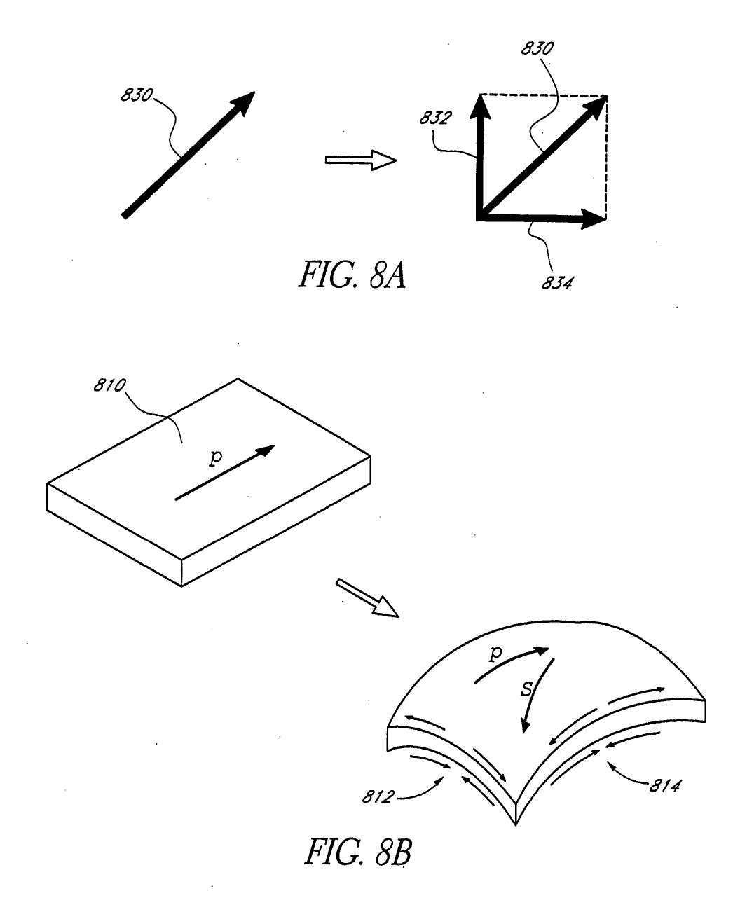

FIG. 8A illustrates a two dimensional vector 830. The vector 830 has a magnitude (corresponding to its length) and a direction in the plane of the page. As illustrated, the vector 830 can be “resolved” into two vector components, the vertical component 832 and the horizontal component 834. The combination of the two components 832 and 834 can provide the same information inherent in the original vector 830. The vertical component 832 and the horizontal component 834 can also be characterized as a “basis set” onto which the vector 830 can be expanded.

Just as this vector 830 can be resolved into components, an incoming signal can be represented as a vector quantity that can be resolved into two components in a Cartesian coordinate system (or another basis set). This concept can be employed to combine two orthogonally polarized (or non-parallel) sensing portions in a single sensor, and from the respective signals of the two sensing portions, directional components can be calculated and an approximation for the signal magnitude can be produced. Thus, if the polarized slabs 710 and 720 are physically combined such that one slab is polarized in a direction that is orthogonal (or non parallel) to the other slab, a device that senses signals coming from any direction can be constructed. Such a device preferably is configured to allow the impinging acoustic signals to arrive at the two slabs essentially in unison—that is, such that the time difference of arrival (TDOA) is minimal. This minimization of the TDOA can be achieved when the sensor comprises two portions of thin PVDF material that partially overlap as illustrated herein.

FIG. 8B illustrates a piezoelectric slab 810 that is undergoing strain that is not aligned with the slab's polarization axis. That is, the strain is in a direction “S” that is neither parallel to nor perpendicular to the polarization axis “P.” The strain “S” can be represented in terms of some orthogonal components 812 (components that are perpendicular to “P”) and some parallel components 814 (components that are parallel to “P”). Under the influence of these the various tensile and contractive forces that result from the strain “S,” the slab 810 does not sense the orthogonal components 812 of these forces as effectively as it senses the parallel components 814. This kind of unaligned strain is typical of that generated by an acoustic signal that originates in the body and arrives at the surface of a patient's skin. Indeed, because of the difficulty in predicting the arrival direction of any acoustic signal to be measured (especially when such a direction may be part of the information sought from the test in the first place), it is unlikely that a sensor's polarity can be perfectly aligned with an incoming acoustic signal. Furthermore, if information is sought relating to a signal's magnitude, but the direction of the signal does not need to be known, a combined system can be sensitive to signals from any direction without a need for aiming the sensor. If the two slabs 710 and 720, in the orientations illustrated in FIG. 7, are combined to form a mechanically coupled system that undergoes the strain illustrated in FIG. 8B, the two sensing portions 710 and 720 can each detect vector components of the acoustic signal. Furthermore, if the two sensing portions 710 and 720 are located in generally the same plane, the TDOA will be minimized and the two sensing portions will be responding to essentially the same signal. Thus, the configuration of sensing portions 210 and 220 illustrated in FIGS. 2-4 can provide multi-directional sensing capability and minimize sensing errors due to a large TDOA.

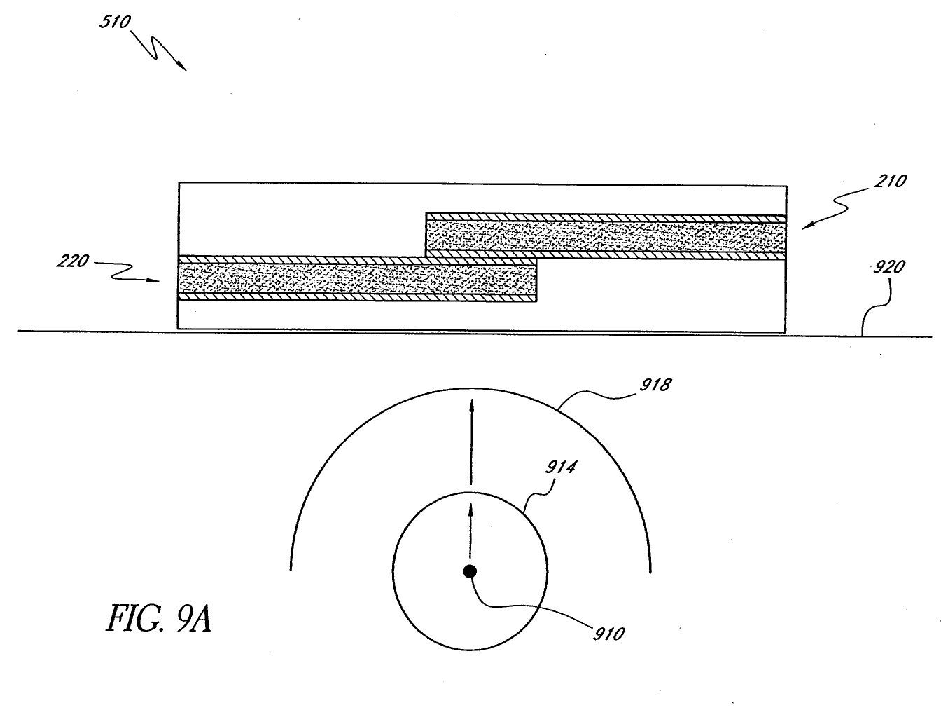

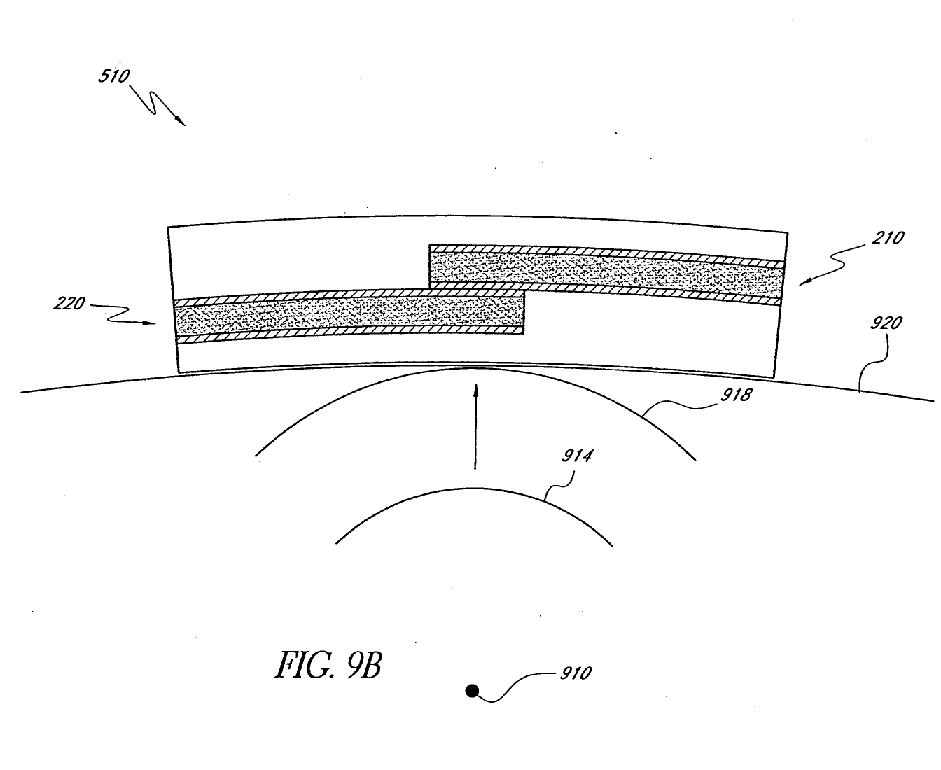

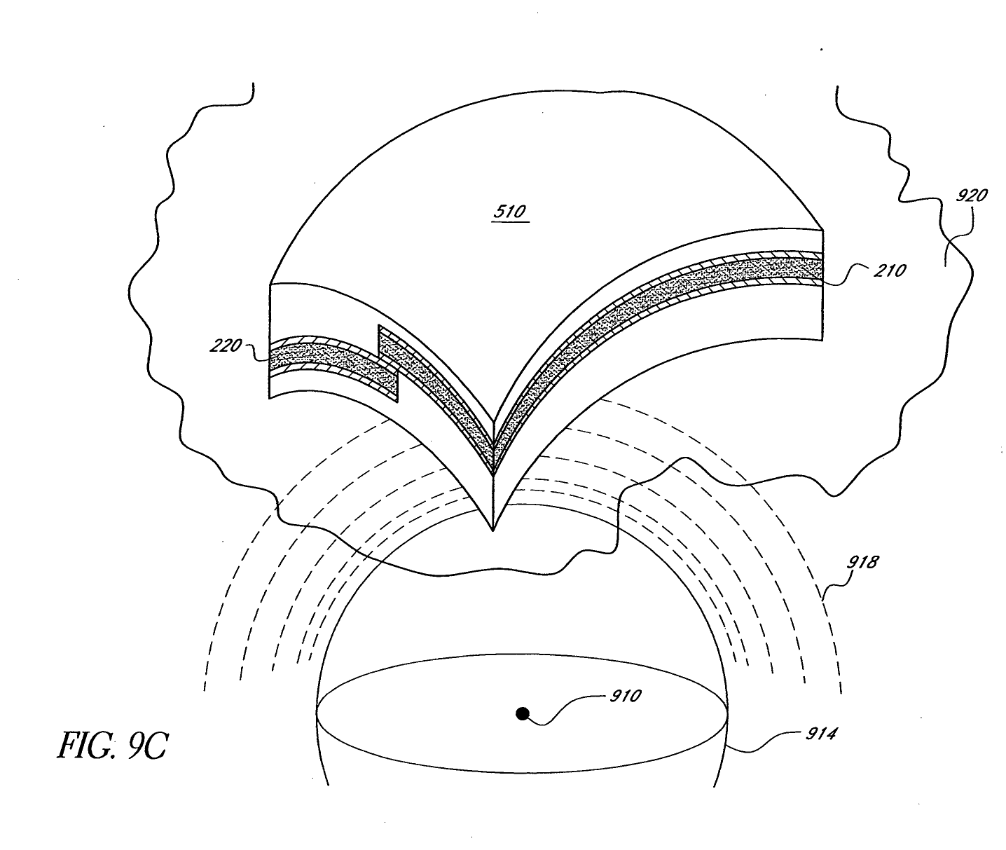

With reference to FIG. 9A, an acoustic source 910 is schematically illustrated, and a circular wavefront 914 is shown emanating from the source 910. Another wavefront 918 is illustrated further away from the source 910, as energy travels upward toward a surface 920. In some embodiments, the source 910 can be located within a human body, and the surface 920 can be the human skin on the surface of the body. A sensor 510 is schematically illustrated, showing sensing portions 210 and 220 within the sensor 510. Before the wavefront 918 arrives at the surface 920, the sensor 510 is not under any significant strain and rests in an equilibrium, essentially zero-signal configuration.

With reference to FIG. 9B, the wavefront 918 has arrived at and deformed the surface 920, which is protruding outward, causing a strain in the surface 920. The sensor 510 is mechanically coupled to the surface 920 and undergoes a proportional strain when the wavefront 918 (corresponding to an acoustic signal) arrives at the surface 920. The strain causes charge to accumulate on the surfaces of the sensing portions 210 and 220, which can in turn cause a signal to be transmitted from the sensor 510 containing information about the magnitude and direction of the wavefront 918.

FIG. 9C schematically illustrates a three-dimensional view of the arrival of the wavefront 918 at the surface 920 depicted in FIG. 9B. As illustrated, the sensing portions 210 and 220 undergo strain in more than one dimension. Thus, the sensing portions 210 and 220 can be polarized in orthogonal directions and the system can gather effective directional information, as described above. This illustrates one advantage of the described sensor embodiments, namely their ability to detect signals from multiple directions. The described characteristics can enhance the sensors' ability to convert acoustical to mechanical energy from acoustic waves that cause of the skin to bend. For example, when it is mounted on human skin, the sensor 510 can receive signals from any direction in the 360° field of view.

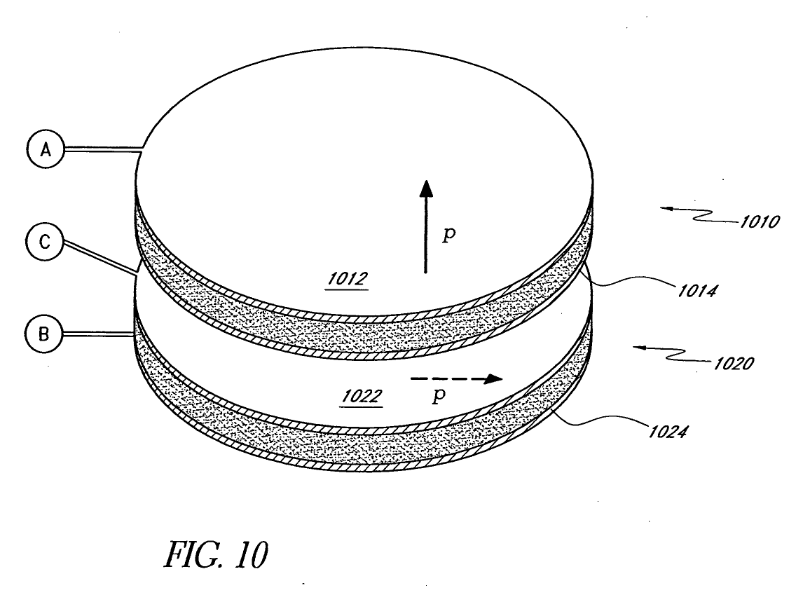

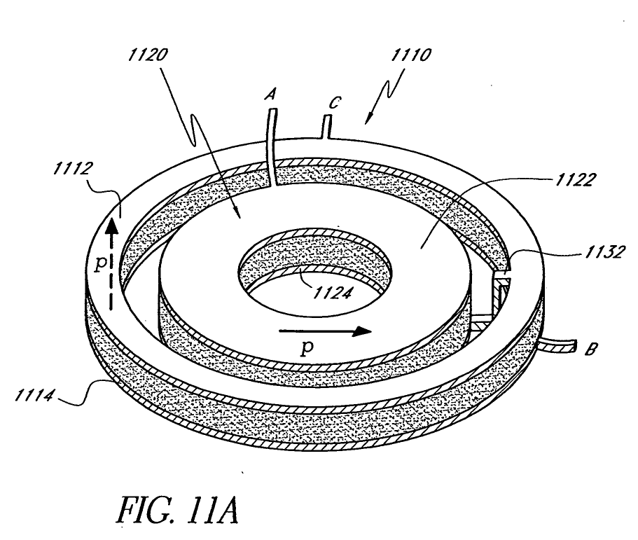

FIG. 10 schematically illustrates an embodiment of two sensing portions 1010 and 1020. The upper sensing portion 1010 has an upper conductive layer 1012 that contacts a lead A, and a lower sensing portion 1014 that is in electrical communication with a lead C. The upper conductive layer 1022 of the lower sensing portion 1020 is also in contact with the lead C, and the lower conductive layer 1024 of the lower sensing portion 1020 is in contact with a lead B. The upper sensing portion 1010 can be positioned to partially overlap the lower sensing portion 1020, as shown. Preferably, the area of the upper and lower sensing portions of any given sensor embodiment are approximately equal. Equal areas simplify the capacitance calculations because the two areas can be cancelled out by dividing both sides of the equation by the area. Thus, in the embodiment illustrated in FIG. 10, the area of the upper sensing portion 1010 is approximately equal to the area of the lower sensing portion 1020. Furthermore, the conductive layers 1012 and 1024 are preferably polarized in one direction, and the conductive layers 1014 and 1022 are preferably polarized in an orthogonal direction, as shown by the polarization arrows labeled “P” in FIG. 10.

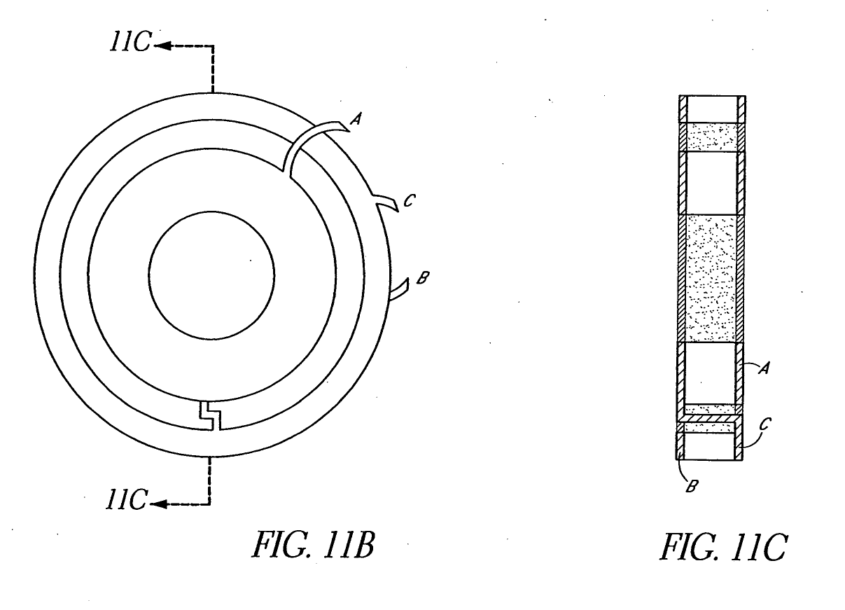

FIG. 11A schematically illustrates a perspective view of an embodiment of two sensing portions, an outer ring 1110 and an inner ring 1120. Preferably, the two rings have the same area, though they do not have the same radius. The outer ring 1110 has an upper conductive layer 1112 that electrically contacts the bottom conductive layer 1124 of the inner ring 1120 through a contact strip 1132. This configuration allows the two sensing portions 1110 and 1120 to be in electrical contact without having any overlapping portions. Furthermore, in this embodiment, the two sensing portions 1110 and 1120 are approximately coplanar. A terminal A is shown to be in electrical contact with the upper conductive layer 1122 of the inner ring 1120. A terminal B is shown to be in electrical contact with the lower conductive layer 1114 of the outer ring 1110. A terminal C is shown to be in electrical contact with the upper conductive layer 1112 of the outer ring 1110, and by extension with the lower conductive layer 1124 of the inner ring 1120.

FIG. 11B schematically illustrates a plan view of the embodiment of FIG. 11A. FIG. 11C schematically illustrates a cross-sectional view taken along the lines 11C-11C of FIG. 11B. The approximately coplanar nature of this embodiment is apparent in the cross-sectional view of FIG. 11C. Furthermore, the conductive layers 1112 and 1124 are preferably polarized in one direction, and the conductive layers 1114 and 1122 are preferably polarized in an orthogonal direction, as shown by the polarization arrows labeled “P” in FIG. 11. The surface areas of the inner and outer rings are preferably equal, which can allow the sensor to achieve a constant gain or signal response when acoustical signals arrive from any direction. This configuration can provide especially efficient data when sensing a spherical wave front because of its general cylindrical symmetry.

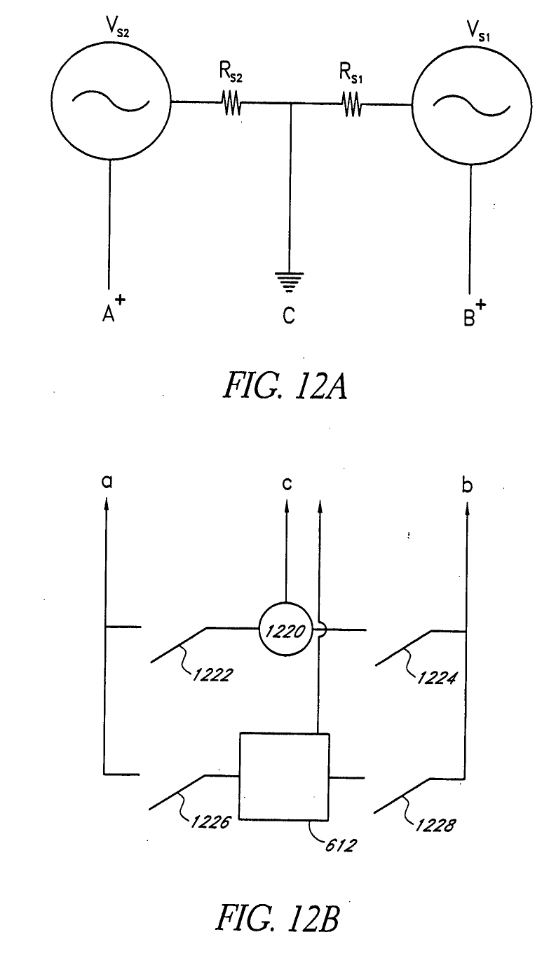

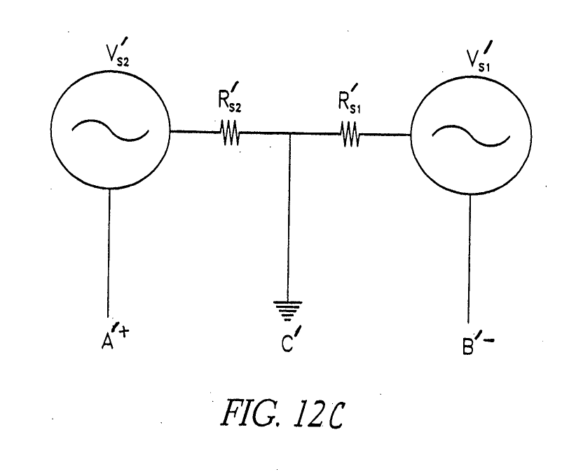

FIG. 12A schematically illustrates an embodiment of an electrical circuit that can be used to represent the electrical response properties of certain of the embodiments discussed above. For example, FIG. 12A may represent the electrical response of embodiments in which the sensing layers 210 and 220 are “inverted” with respect to each other. A terminal A is connected to a variable voltage source V_{S2}. The variable voltage source V_{S2} can correspond to the conductive layers 212, 1012, or 1122, for example. A terminal B is connected to a variable voltage source V_{S1}. The variable voltage source V_{S1} can correspond to the conductive layers 224, 1024, or 1114, for example. The conductive layers have variable voltages that depend on the amount of strain on (and charge that accumulates on the surfaces of) the piezoelectric portions with which they are in electrical contact. A terminal C connects the opposite sides of the two variable voltage sources—through resistances R_{S1} and R_{S2}—to ground. In practical effect, the resistances R_{S1} and R_{S2} generally approach zero. The C terminal can correspond to the conductive layers 214 and 222, 1014 and 1022, and 1112 and 1124, for example. When these layers are connected to ground, they can draw (or deposit) as much charge as needed to balance out the charge that flows to terminals A and B as a result of the piezoelectric effect on the sensing portions. The voltage difference across terminals A and C is measured, and the voltage difference across terminals C and B is measured. In some embodiments, the A-C voltage can provide information relating to the signal corresponding to one vector component of the impinging acoustic signal, and the B-C voltage provides information relating to the other vector component. FIG. 12C schematically illustrates another embodiment of an electrical circuit that can be used to represent the electrical response properties of certain other embodiments discussed above. For example, FIG. 12C may represent the electrical response of embodiments in which the sensing layers 210 and 220 are not “inverted” with respect to each other. Generally as described above with reference to FIG. 12A, in some embodiments the A′-C′ voltage can provide information relating to the signal corresponding to one vector component of the impinging acoustic signal, and the B′-C′ voltage provides information relating to the other vector component.

FIG. 12B schematically illustrates an embodiment of an electrical circuit that can be used to test the circuit illustrated in FIG. 12A and/or analyze the data produced by the embodiments described above. The terminal a can be connected to A, the terminal c can be connected to C, and the terminal b can be connected to B. If the switch 1222 is closed, a multimeter 1220 can measure the current, resistance, and/or voltage drop across terminals A and C. If the switch 1224 is closed, the multimeter 1220 can measure the current, resistance, and/or voltage drop across terminals C and B. Similarly, if the switches 1226 and 1228 are both closed, the signal processor 612 can process the signals provided by the sensor to determine electrical and acoustic characteristics of the sensed system.

Embodiments of acoustic sensors described herein advantageously provide increased sensitivity to acoustic signals. However, in some instances, it may be desirable to reduce acoustic noise received by a sensor in order to provide a more accurate, precise, and/or higher signal-to-noise measurement of a particular acoustic signal. “Acoustic noise” or “noise” are broad terms and are used in their ordinary sense and can include ambient, environmental, and/or background noise and/or vibrations that are transmitted to the vicinity of the acoustic sensors. Acoustic noise can include, for example, distracting background noises and chatter that are detectable by a human ear as well as low-amplitude, unheard vibrations caused by, e.g., a distant truck driving down the street, the hum of electrical transformers, ballasts, and motors, etc.

Acoustic noise received by an acoustic sensor can contribute to the deformation of portions of the piezoelectric acoustic sensing layers and thereby contribute to the electrical signal output by the sensor. Although there are various signal processing methods (e.g., filters) that can be applied to post-process a sensor signal so as to partially remove acoustic noise artifacts, it is beneficial in some instances to provide noise reduction features that reduce the intensity of acoustic noise received by the sensing layers. For example, acoustic noise may be noise that originates from one location or one direction. A directional shield or damper can be used to impede that noise from reaching the sensor, while acoustic signals from other directions can be allowed to proceed, unobstructed. Some embodiments of an acoustic sensor having noise reduction features thereby beneficially produce a cleaner and less noisy electrical output signal, which can enable, for example, higher signal-to-noise measurements of relatively faint sounds in an acoustically noisy environment.

As used herein, “noise reduction” and “noise reducing” are general terms and are used in their broad sense to mean components, devices, and elements that reduce the amount (e.g., amplitude, energy, intensity, flux, etc.) of acoustic noise that is received by the sensor and/or by any acoustic sensing elements disposed in the sensor. Noise reduction features may include, for example, features that shield and/or dampen acoustic noise. Generally, acoustic dampening features attenuate or absorb acoustic noise as the noise propagates toward the sensing elements. Generally, acoustic shielding features deflect, reflect, and/or refract acoustic noise to reduce or prevent the noise from reaching the sensing elements. As used herein, noise reducing elements can utilize shielding, dampening, and/or any other known (or presently unknown) effect to reduce acoustic noise received by the acoustic sensors. For example, various noise reducing elements may use a combination of shielding, dampening, interference, diffraction, etc. to provide suitable noise reduction.

Acoustic sensors having noise reduction features are advantageously used, for example, in systems configured to detect and analyze sounds produced by the human body (e.g., the heart sound detection systems described with reference to FIG. 6). Sounds produced by the human body often have intensities that are small compared with the typical intensity of acoustic noise in an examining room. Moreover, in certain instances, only a portion of the acoustic signal carries diagnostic information related to a medical condition. For example, some embodiments of the systems described in U.S. patent application Ser. No. 10/830,719 and U.S. patent application Ser. No. 11/333,791 utilize portions of heart sound signals having frequencies in a band from about 300 Hz to about 2000 Hz. Since the acoustic intensity of a diagnostically useful portion of an acoustic signal is generally smaller than the intensity of the complete acoustic signal, it is particularly beneficial in such systems to utilize acoustic sensors that can reduce acoustic noise received by sensing elements.

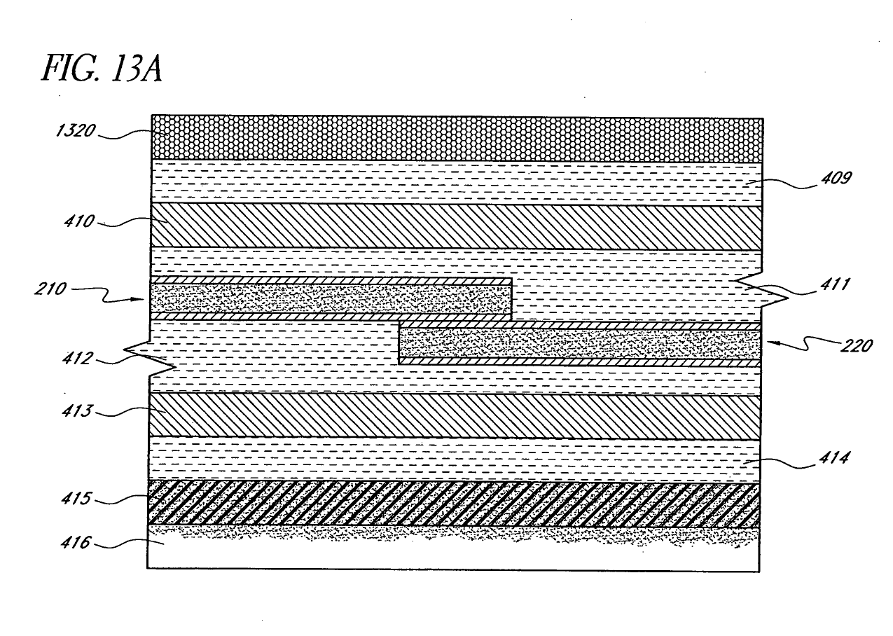

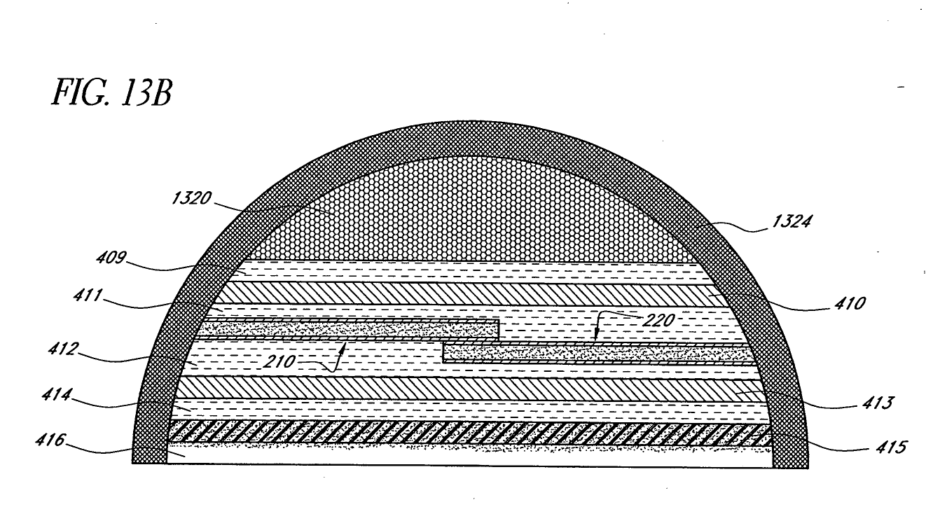

FIG. 13A is a schematic, partial cross-sectional side view of a portion of a noise reducing acoustic sensor. The illustrated sensor is generally similar to the sensor described with reference to FIG. 4 except as further described herein, and like reference numerals correspond to generally similar features. The embodiment of the acoustic sensor shown in FIG. 13A comprises one or more acoustic noise reduction layers 1320. Generally, the noise reduction layer 1320 is disposed on or adjacent to at least a portion of at least one surface of the sensor. In certain embodiments, the noise reduction layer 1320 substantially surrounds the acoustic sensor except for a portion of the sensor that is configured to be attached or otherwise in acoustic contact with the object to be sensed. For example, in the embodiment shown in FIG. 13A, the acoustic sensing layers 210 and 220 are interposed between the compliant membrane 415 and the noise reduction layer 1320. The noise reduction layer 1320 is attached to the sensor by the adhesive material 409 in certain embodiments. The compliant membrane 415 can be attached to the skin of a patient via the biocompatible adhesive 416 so as to receive acoustic body signals. The noise reduction layer 1320 advantageously reduces ambient acoustic noise received by the sensing layers 210, 220 but does not significantly reduce the acoustic signals received from the body of the patient (e.g., through the compliant membrane 415).

For purposes of illustration only, FIG. 13A schematically illustrates an embodiment of the sensor comprising a single noise reduction layer 1320; however, this is not intended to be a limitation. In other embodiments, more than one noise reduction layer can be used, for example, two, three, four, or more layers. The layers may be separated from each other (and/or from other components in the sensor) via any type of suitable material or substance including, for example, adhesives, gaps (including air gaps and/or gaps with partial or reduced pressure), electrical shielding layers (e.g., metal layers 410, 413), etc. In some embodiments, the noise reduction layer 1320 is attached to the sensor via an adhesive material 409. In certain embodiments, the noise reduction layers are spaced apart from other layers in the sensor so as to provide increased acoustic dampening in regions where the acoustic velocity is relatively large (e.g., at an antinode). In other embodiments, the configuration of the noise reduction layers is different from that shown in FIG. 13A. For example, in some embodiments, the layers are not plane parallel layers, and may have any suitable shape, thickness, and/or size.

The acoustic noise reduction layer 1320 may comprise any type of material suitable to reduce acoustic waves propagating into or through the layer 1320. In some embodiments, the acoustic noise reduction material comprises an open cell foam (e.g., polyurethane, polyester, polyether, melamine, etc.), elastomers (e.g., soft silicone), gels, composites of randomly adhered elastomeric particles, viscoelastic materials, or other suitable material combinations of any of the foregoing can also be used. For example, the layer 1320 can be formed of a thin elastomer skin surrounding a gel. The materials may be selected to provide increased noise reduction for acoustic waves having certain frequencies. For example, in some embodiments, the noise reduction layer 1320 is selected to reduce sound waves in the frequency band that includes the sounds to be detected and/or analyzed. In an embodiment suitable for use with heart sound detection systems, for example, the frequency band of interest can extend from about 300 Hz to about 2000 Hz, and the noise reduction layer can be selected or designed to attenuate acoustic signals generally within that frequency band. In some embodiments, the acoustic noise reduction layer 1320 comprises a multilayer laminate designed to attenuate a wider band of acoustic frequencies, for example, from a few Hz to 10 kHz. In certain embodiments, such as heart sound detection systems, it is preferable for the noise reduction layer 1320 to comprise a material that is sufficiently flexible so that the sensor can conform to the contours of the patient's skin.

The noise reduction layer 1320 is selected, in some embodiments, to provide a desired amount of attenuation of the acoustic noise. For example, in certain embodiments, the noise reduction layer is configured to provide, e.g., 3 dB, 5 dB, 10 dB, 20 dB, 30 dB of acoustic attenuation. In some embodiments, the acoustic noise reduction layer 1320 has a thickness in a range from about 1 mm to about 3 cm. In one preferred embodiment, a layer of soft silicone with a thickness of about ⅛ inch is used. Depending on the softness of the silicone, the layer thickness can be different (e.g., softer silicone may require larger thickness to achieve the same attenuation).

In some embodiments, the noise reduction layer comprises material that acts, in part, to shield, deflect, reflect, or refract sound waves away from the sensing layers 210 and 220. For example, in the embodiment schematically illustrated in FIG. 13B, a noise reduction layer comprises a shell 1324, such as a semi-rigid shell, that substantially surrounds the other portions of the acoustic sensor. In such embodiments, the shell 1324 partially shields the sensing layers 210, 220 from acoustic noise energy by, for example, preventing a substantial portion of the incident acoustic noise from reaching the sensing layers 210, 220. The shell 1324 in some embodiments is shaped so as to shield acoustic noise from a reasonably wide range of incident directions. For example, the shell 1324 shown in FIG. 13B has a rounded shape (e.g., a portion of a sphere, ovoid, ellipse, etc.); however, other shapes can be used. For example, in some embodiments, a shell can have one or a plurality of flat or angled surfaces. In some embodiments, the shell is spaced apart from the other layers of the sensor by an air gap, an acoustic dampening foam, or other suitable material to provide further attenuation of any acoustic noise signals transmitted by the shell. In the embodiment shown in FIG. 13B, an acoustic dampening layer 1320 is disposed between the shell 1324 and the other layers. As described further herein, the sensor can be applied to the skin via a biocompatible adhesive layer 416. The sensor embodiment shown in FIG. 13B advantageously both shields and dampens ambient noise so as to substantially reduce the noise contributions which reach the positions of the sensing layers 210 and 220.

Although several embodiments of a sensor having noise reduction functionality have been described, it will be apparent to a skilled artisan that alternative materials, structures, components, and configurations can be used without departing from the scope or the spirit of the inventions. In various embodiments, the noise reduction components can be configured to provide a greater or lesser degree of noise reduction as reasonably needed, or in certain embodiments, the noise reduction components can be eliminated. Many variations are possible.

The present disclosure describes various embodiments of acoustic sensors that provide improved sensitivity, improved electrical and acoustic shielding, reduced differences in acoustic time arrival, and increased signal-to-noise. Such sensors advantageously can be used to detect low amplitude acoustic signals from the human body. As described above with reference to FIG. 6, embodiments of any of the acoustic sensors disclosed herein provide benefits when used with a system to detect heart sounds from a patient. In some of these systems, it is desirable to identify one or more portions of the heartbeat such as, for example, the diastolic or systolic portions. For example, U.S. patent application Ser. No. 10/830,719 and U.S. patent application Ser. No. 11/333,791 disclose embodiments of systems in which electrocardiogram (“EKG”; also known as “ECG”) signals can be used in conjunction with acoustic heartbeat signals to detect, diagnose, and/or locate occlusions in coronary arteries. Some embodiments of these systems utilize separate acoustic sensors and EKG sensors to detect the acoustic and electrical signals, respectively, from the heart. However, embodiments that use separate sensors for the acoustic and the electrical signals may require attaching up to 16 sensors to the patient (e.g., 4 acoustic sensors and 12 EKG sensors in one system). Accordingly, a single sensor that combines both acoustic and electrical sensitivity advantageously can not only reduce the number of sensors that must be attached to the patient but can also increase patient comfort and reduce the likelihood that one or more nonfunctioning sensors will cause spurious measurements.

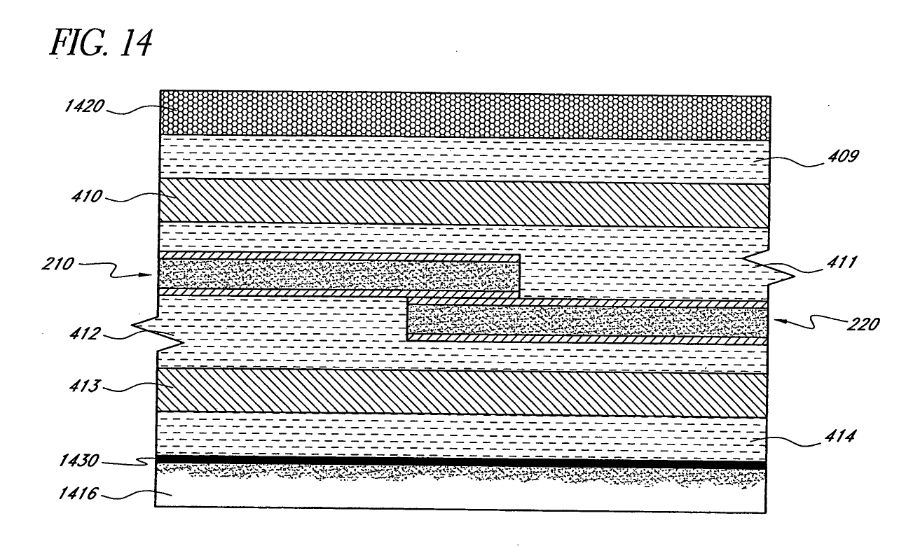

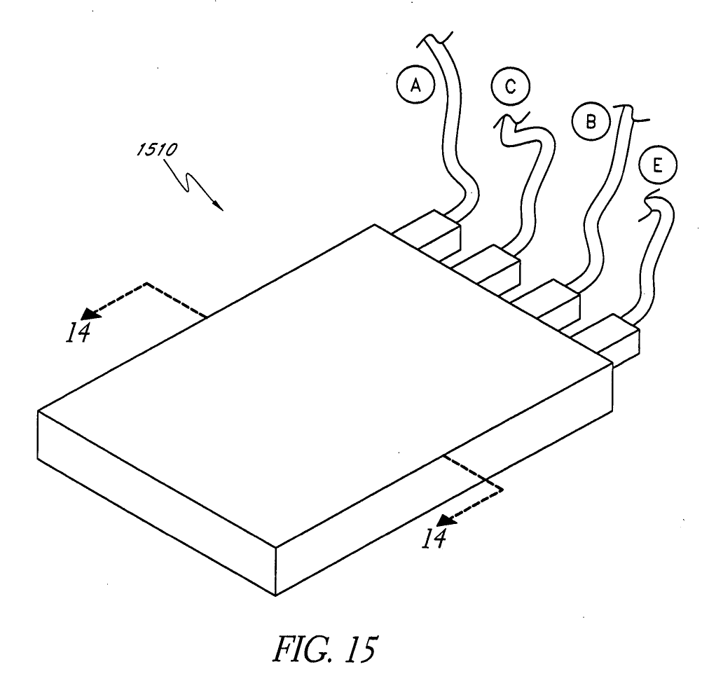

FIG. 14 is a schematic, partial cross-sectional side view of a portion of a combined acoustic and electrical sensor in accordance with one embodiment of the inventions. The side view is taken along the line 14-14 in FIG. 15. The sensor is generally similar to the sensors described with reference to FIG. 4 and FIG. 13 except as further described herein, and like reference numerals correspond to generally similar features. In this embodiment, the sensor includes an acoustic noise reduction layer 1420 that is generally similar to the layer 1320 shown in FIG. 13A. In other embodiments, other noise reducing features are included such as, for example, an acoustic noise shielding layer (e.g., the shell 1324 shown in FIG. 13B). However, in other embodiments, the sensor comprises a compliant membrane (generally similar to the membrane 417 in FIG. 4) that is used in addition to or instead of the noise reduction layer 1420.

In order to detect and transmit electrical signals, the combined acoustic and electrical sensor comprises an electrically conductive electrode 1430. As shown in FIG. 14, the combined sensor can be configured so that the electrode 1430 can be attached or otherwise electrically coupled to the patient's skin (e.g., the electrode 1430 is the bottom or lowest layer in the sensor). In some embodiments, an electrically conductive adhesive 1416 can be placed on the electrode 1430 prior to placement on the skin. Preferably, the electrically conductive adhesive 1416 comprises a biocompatible substance having an electrical impedance that is sufficiently small to reduce resistive losses and an acoustic impedance to reduce acoustic reflection losses. In some embodiments, the adhesive 1416 is selected to have an acoustic impedance that approximately matches that of the body. The adhesive 1416 in some embodiments comprises an electrically conductive hydrogel, which can be positioned at the skin-sensor interface to improve sensitivity and acoustic/mechanical/electrical coupling. In some embodiments, the electrically conductive adhesive 1416 is smeared onto the human skin surface where the sensor will be placed, and the sensor is then pressed onto the same area of the skin. The adhesive 1416 can also be placed on the sensor before it is pressed into place. In some embodiments, the electrically conductive adhesive 1416 is located beneath a removable strip or tab (not shown) on the sensor when the sensor is packaged, and the user can remove the strip to reveal the biocompatible adhesive 1416 underneath, immediately prior to using the sensor.

In certain embodiments the electrode 1430 comprises a thin, electrically conductive layer configured to be electrically connected to (or positioned on or near) the patient's skin. The electrode 1430 can be adhered to the sensor by, e.g., an adhesive layer 414. Preferably, the adhesive layer 414 is electrically nonconductive so as to insulate the electrode 1430 from the metal shielding layers 410, 413 and from the electrically conductive portions of the sensing layers 210, 220. In some embodiments, the electrode 1430 comprises one or more metal layers, such as aluminum, nickel, silver, gold, copper, or alloys or salts thereof. Although in some embodiments the metal layer is adhered to the sensor by the adhesive layer 414, in other embodiments, the metal layer is formed on a lower surface of the sensor by, e.g., coating, metallization, vapor deposition, or other methods. In such embodiments, an insulating dielectric layer or nonconductive substance may be interposed between the electrode 1430 and the metal shielding layer 413. In other embodiments, the electrode 1430 can be coated on the lower surface of the sensor with a conducting ink. In a preferred embodiment, the electrode 1430 comprises one or more layers of an electrically conductive plastic, polymer, or composite, such as, for example, polyether or polyester urethane containing conductive carbon additives. In some embodiments the electrode 1430 comprises a multilayer laminate comprising alternating layers of electrically conductive plastic and metal to increase the electrical conductivity.

It is preferable, although not necessary, for the electrode 1430 to be sufficiently thin and flexible so that it can conform to the contours of the patient's skin. Moreover, a sufficiently thin and flexible electrode 1430 advantageously can permit incident acoustic signals to propagate into the interior of the combined sensor so as to be detected by the acoustic sensing layers 210 and 220. In some preferred embodiments, the electrode 1430 has a thickness less than or equal to about 30 μm, although other thicknesses can be used (e.g., from about 10 μm to about 100 μm). In other embodiments, the thickness of the electrode can be up to or equal to about 1 mm, or more.

In the embodiment shown in FIG. 14, the electrode 1430 is disposed below and slightly spaced apart from the sensing layers 210 and 220. Preferably the thickness of the spacing between these layers is sufficiently small that there is minimal difference between the arrival time of electrical signals (at the electrode 1430) and the acoustic signals (at the sensing layers 210, 220). Accordingly, the combined sensor preferably makes simultaneous or nearly simultaneous measurements of the electrical and acoustical activity in the patient's body. In other embodiments, the electrode 1430 may be disposed differently than shown in FIG. 14. For example, in some embodiments, the electrode 1430 is not disposed below the other layers shown in FIG. 14 but is laterally displaced from and/or partially surrounds a portion of a perimeter or a circumference of the acoustically sensitive layers 210, 220. In embodiments combining an electrode with the acoustic sensor shown in FIG. 11A, the electrode comprises electrically conductive material may be disposed in the annulus within the inner ring 1120, and/or within the annular gap between the inner ring 1120 and the outer ring 1110, and/or outside the outer ring 1110. In other embodiments, the electrode comprises electrically conductive material disposed in one or more layers disposed below the inner and/or outer rings 1120, 1110. Certain embodiments of the combined acoustic sensor in which the electrode is not disposed below the acoustic sensing layers 210 and 220 advantageously permit acoustic and electrical signals to be received substantially simultaneously by the acoustic sensing layers and the electrode, respectively. Moreover, in some of these embodiments, acoustic signals from a source within the body advantageously reach the acoustic sensing layers without having to propagate through the electrode.

In certain preferred embodiments, the electrode 1430 functions as an EKG electrode and can be used to measure electrical signals such as, for example, the action potentials produced by the contraction and relaxation of the heart muscle. As is well known, during the heartbeat cycle electrical currents spread through the body and create differing electrical potentials on the skin. By placing one or more electrodes 1430 in electrical contact with the skin, these potential differences can readily be measured by techniques that are well known in the medical arts. Generally, an EKG detects electrical potential (e.g., voltage); however, the electrode 1430 is not so limited and can be used to detect, for example, electric current, resistance, or other suitable quantity. In various systems, between two and twelve electrical sensors are used to measure the heart's electrical signals (which have peak-to-peak amplitudes of about 1 mV). In some embodiments, any metal layer can function as an EKG electrode. For example, in some embodiments, layers such as the metallic shielding layers 410 and 413 can function to measure electrical signals.

The normal heartbeat cycle comprises a number of electrical features such as a P wave, a QRS complex, and a T wave. The QRS complex represents the contraction of the ventricles, with the R wave typically being the feature with the largest electrical potential. Certain embodiments of systems for detecting and processing heart sounds, such as those described in U.S. patent application Ser. No. 10/830,719 and U.S. patent application Ser. No. 11/333,791, utilize signatures in the electrical heart signals (such as, e.g., the duration between successive R-waves) for diagnostic purposes such as to identify the heart rate (in beats per minute) and the duration of the diastolic and/or systolic portions of a heartbeat. The combined acoustic and electrical sensor described herein can advantageously be utilized with these heart sound systems, because the combined sensor measures both the acoustic and the electrical signals emitted by the heart. The use of combined sensors beneficially reduces the total number of sensors needed to measure and diagnose heart sounds (e.g., four combined sensors are used in one embodiment). Further details regarding the placement of the sensors on a patient's body can be found in, for example, U.S. patent application Ser. No. 10/830,719 and U.S. patent application Ser. No. 11/333,791. In other embodiments, the combined sensors may be placed according to other principles of electrocardiography (e.g., according to Einthoven's triangle).

FIG. 15 is a schematic perspective view of a combined acoustic and electrical sensor 1510 in accordance with one embodiment of the inventions. The combined sensor 1510 preferably can flex so that it conforms to the patient's skin and so that the sensing layers 210 and 220 within the sensor 1510 can be deformed so as to produce electrical signals in response to incident acoustic energy as described above. Additionally, a portion of the sensor 1510 (e.g., the electrode 1430) preferably is in electrical contact with the skin so as to produce an electrical signal indicative of the electrical potential at the point of contact. It is desirable that the electrode 1430 in the sensor 1510 be sufficiently thin and flexible so that acoustic signals from the body are not substantially attenuated as they pass through the electrode 1430 and so that the electrical and acoustical signals have a sufficiently small difference of arrival time.

As schematically illustrated in FIG. 15, the sensor 1510 comprises four electrical leads: A, B, C, and E. The three leads A, B, and C are generally substantially similar to the leads A, B, and C described with reference to FIG. 5 and are used to provide electrical connectivity to the conductive layers in the acoustic sensing layers 210 and 220. The electrical lead labeled E in FIG. 15 provides electrical connectivity to the electrode 1430. The lead E can comprise any suitable type of electrical connection as is well known in the art. For example, the electrical lead E can comprise a metal lug connected to (or formed as part of) the electrode 1430. In one embodiment, the E lead has a 5 mm lip. In some embodiments, the E lead is attached to a shielded pair of twisted wires generally similar to the wires 248 described with reference to FIG. 3. The E lead can be connected to the wires by soldering, crimping, an EC adhesive, or some other suitable method.

The E lead shown in FIG. 15 carries electrical signals detected by the electrode 1430 to other devices or components for further processing or measurement. For example, in one embodiment, the E lead is connected to a multimeter (which can be generally similar to the multimeter 1220 in FIG. 12B), which can measure suitable electrical quantities such as current, resistance, and/or voltage. In other embodiments, the E lead may be connected (alternatively or additionally) to a processor such as the signal processing system 612 shown in FIG. 6. In such embodiments, the electrical signal may be processed by one or more well-known techniques including, for example, digitizing, filtering, amplifying, and/or multiplexing. Electrical signals detected by the electrode 1430 may be used by body sound detection systems (including the systems disclosed in U.S. patent application Ser. No. 10/830,719 and U.S. patent application Ser. No. 11/333,791) for purposes such as identifying desired portions of a heartbeat or for other suitable purpose. In some advantageous embodiments, systems utilize signals from both the electrode 1430 and the acoustic sensing layers 210, 220 to perform diagnostic procedures.

Although some advantageous embodiments of sensors having noise reduction features and/or electrical detection features have been described, it is recognized that other embodiments may be configured differently from those schematically illustrated in FIGS. 13A-15. For example, a person of ordinary skill will appreciate that noise reduction and electrical features can readily be incorporated into the sensor embodiments described above with reference to FIGS. 10 and 11A-11C. Moreover, the functions of some of the layers described above can be combined, in some embodiments. For example, an adhesive function and a noise attenuation function may be accomplished (or aided) by a single tacky foam layer. Thus, many layer combinations and/or sensor configurations are possible without departing from the scope or the spirit of the present disclosure.

The embodiments described herein can advantageously be adjusted for different (e.g., highly efficient) manufacturing processes. For example, electrical connections and circuits can be formed using chemical deposition and integrated circuit processes. Moreover, materials can be deposited, one onto another, in a form and using a deposition process that eliminates the need for the adhesive materials described herein. A person of ordinary skill will recognize that the sensors described herein can be fabricated using a variety of manufacturing methods and techniques without departing from the scope of these inventions.

Although the present inventions have been described in terms of certain preferred embodiments, various features of separate embodiments can be combined to form additional embodiments and obvious modifications not expressly described. Moreover, other embodiments apparent to those of ordinary skill in the art after reading this disclosure are also are within the scope of these inventions. Further, although various features, aspects, and advantages have been described herein where appropriate, it is recognized that not every embodiment need incorporate or achieve each such feature, aspect, or advantage. Thus, for example, certain embodiments may achieve or optimize one advantage or group of advantages as taught herein without necessarily achieving other features, aspects, or advantages as taught or suggested herein. Various changes, modifications, and combinations may be made without departing from the spirit and scope of the inventions. Accordingly, it is intended that the scope of the inventions disclosed herein should not be limited by the particular embodiments described above, but should be determined by a fair reading of the claims that follow.

What is claimed is:

1. An apparatus for sensing acoustic signals from a source, the apparatus comprising:
a first acoustic sensing element comprising a first piezoelectric portion having a first polarization axis and two electrically conductive portions;
a second acoustic sensing element comprising a second piezoelectric portion having a second polarization axis and two electrically conductive portions; and
a noise reducing element;
wherein one electrically conductive portion of the first piezoelectric portion is electrically connected to one electrically conductive portion of the second piezoelectric portion,
wherein the first and the second piezoelectric portions are oriented such that the first and the second polarization axes form a non-zero angle therebetween.

2. The apparatus of claim 1, wherein the noise reducing element comprises a noise reducing material adapted to attenuate acoustic noise.

3. The apparatus of claim 2, wherein the noise reducing material comprises a noise reducing foam, elastomer, or gel.

4. The apparatus of claim 2, wherein the noise reducing material substantially surrounds at least a portion of at least one of the first and the second acoustic sensing elements.

5. The apparatus of claim 1, wherein the noise reducing element is configured to at least partially shield at least one of the first or the second acoustic sensing elements from acoustic noise.

6. The apparatus of claim 5, further comprising a noise reducing material adapted to attenuate acoustic noise.

7. The apparatus of claim 1, wherein the noise reducing element is configured to reduce acoustic noise in an acoustic frequency band.

8. The apparatus of claim 7, wherein the acoustic frequency band is from about 300 Hz to about 2000 Hz.

9. The apparatus of claim 1, wherein at least a portion of the noise reducing element is spaced apart from the first and the second acoustic sensing elements.

10. The apparatus of claim 1, further comprising a housing, the first and the second acoustic elements disposed in the housing, the housing comprising a surface portion adapted to permit acoustic signals from the source to be received by the first and the second sensing elements.

11. The apparatus of claim 10, wherein the noise reducing element comprises noise reducing material disposed within the housing.

12. The apparatus of claim 11, wherein at least a portion of the first or the second acoustic sensing elements is disposed generally between the surface portion of the housing and the noise reducing material.

13. The apparatus of claim 11, wherein the noise reducing material substantially surrounds the first or the second acoustic sensing element.

14. The apparatus of claim 10, wherein the source is located in a body and the surface portion of the housing is adapted to applied to the skin of the body.

15. The apparatus of claim 1, wherein the non-zero angle is about ninety degrees.

16. An apparatus for sensing acoustic and electric signals, the apparatus comprising:
a first acoustic sensing element comprising a first piezoelectric portion having a first polarization axis and two electrically conductive portions;
a second acoustic sensing element comprising a second piezoelectric portion having a second polarization axis and two electrically conductive portions; and
an electrode electrically insulated from the electrically conductive portions of the first and the second acoustic sensing elements,
wherein one electrically conductive portion of the first piezoelectric portion is electrically connected to one electrically conductive portion of the second piezoelectric portion,
wherein the first and the second piezoelectric portions are oriented such that the first and the second polarization axes form a non-zero angle therebetween.

17. The apparatus of claim 16, wherein the electrode comprises an electrically conductive layer.

18. The apparatus of claim 17, wherein the electrically conductive layer comprises a metal or a metal alloy.

19. The apparatus of claim 16, wherein the electrode is configured to at least partially transmit acoustic signals from an acoustic source so that the acoustic signals can be received by at least one of the first or the second acoustic sensing elements.

20. The apparatus of claim 16, wherein the electrode is adapted to be electrically coupled to the skin of a patient.

21. The apparatus of claim 20, wherein the electrode is adapted to receive electrical signals from the heart of the patient and the first and the second acoustic sensing layers are adapted to receive acoustic signals from anatomical structures within the patient.

22. The apparatus of claim 16, further comprising a noise reducing element.

23. The apparatus of claim 22, wherein the noise reducing element comprises a noise reducing material adapted to attenuate acoustic noise.

24. The apparatus of claim 22, wherein the noise reducing element is configured to at least partially shield at least one of the first or the second acoustic sensing elements from acoustic noise.

25. The apparatus of claim 16, wherein the non-zero angle is about ninety degrees.
